(19) United States
(12) Patent Application Publication
(10) Pub. No.: US 2010/0041973 A1
(43) Pub. Date: Feb. 18, 2010

(54) CATHETER RADIO FREQUENCY ADAPTER FOR WIRELESS COMMUNICATION

(76) Inventors: William Minh VU, Laguna Niguel, CA (US); Tho Hoang NGUYEN, Huntington Beach, CA (US)

Correspondence Address: MATTINGLY & MALUR, P.C., 1800 DIAGONAL ROAD, SUITE 370, ALEXANDRIA, VA, 22314, US

(21) Appl. No.: 12/508,313

(22) Filed: Jul. 23, 2009

Related U.S. Application Data
(60) Provisional application No. 61/135,837, filed on Jul. 23, 2008.

(51) Int. Cl. A61B 5/042

(52) U.S. Cl. 600/374

(57) ABSTRACT

A catheter system for wireless communication with an electrophysiological (EP) mapping system. The catheter system comprises a catheter, a catheter adapter, and a radio frequency receiver module. The catheter includes a plurality of mapping electrodes including a tip electrode disposed on a distal portion of the elongated body, the mapping electrodes detecting electrocardiograph (ECG) signals; and a reference electrode being disposed on the elongated body at a distance from the plurality of mapping electrodes such that the reference electrode substantially does not detect electrocardiograph signals. The catheter includes a handle. The catheter adapter is attached to the handle. The catheter adapter includes an RF transmitter module for receiving, processing, and transmitting the detected ECG signals. The reference electrode provides a reference signal to the radio frequency (RF) transmitter module. The RF receiver module receives the transmitted ECG signals. The RF receiver module is coupled to the EP mapping system.

### CROSS REFERENCE TO RELATED APPLICATIONS

This application claims the benefit of U.S. Provisional Application Ser. No. 61/135,837, filed on Jul. 23, 2008, entitled “Catheter radio frequency adapter”, which is hereby incorporated by reference in its entirety.

### BACKGROUND OF THE INVENTION

This invention relates generally to electrophysiological (EP) mapping systems and catheter devices, and more specifically to a radio frequency (RF) adapter for providing wireless communication between a catheter and an electrophysiological mapping system.

Catheters are flexible, tubular devices that are widely used by physicians performing medical procedures to gain access into interior regions of the body. For diagnostic purposes, a catheter is usually connected by a cable to an EP mapping system. The catheter includes a plurality of electrodes on its distal area. The catheter electrodes detect signals from the tissue surrounding the distal area of the catheter and send the detected signals to the EP mapping system. The EP mapping system uses the detected signals to generate a map of the tissue surrounding the catheter distal region.

Currently, a catheter cannot communicate wirelessly with an EP mapping system.

### BRIEF SUMMARY OF THE INVENTION

One embodiment of the present invention is a catheter system for wireless communication with an electrophysiological (EP) mapping system. The catheter system comprises a catheter, a catheter adapter, and a radio frequency receiver module. The catheter includes an elongated body having a distal end, and a proximal end, a plurality of mapping electrodes including a tip electrode being disposed on a distal portion of the elongated body, the plurality of mapping electrodes detecting electrocardiograph (ECG) signals; and a reference electrode being disposed on the elongated body at a distance from the plurality of mapping electrodes such that the reference electrode substantially does not detect electrocardiograph (ECG) signals. The catheter includes a handle. The catheter adapter is attached to the handle. The catheter adapter includes an RF transmitter module for receiving, processing, and transmitting the detected ECG signals. The reference electrode provides a reference signal to the radio frequency (RF) transmitter module. The RF receiver module receives the transmitted ECG signals. The RF receiver module is coupled to the EP mapping system.

### BRIEF DESCRIPTION OF THE DRAWINGS

FIG. 1 is a block diagram illustrating the system 100 of the present invention.

FIG. 2 is a block diagram of one embodiment of the RF transmitter module 120 of the present invention.

FIG. 3 is a block diagram of one embodiment of the RF receiver module 130 of the present invention.

FIG. 4 is a block diagram illustrating the self-creating reference scheme of the present invention.

FIG. 5 shows an external view of an embodiment of the catheter 110.

FIG. 6 shows an embodiment 600 of the system of the present invention, where a single receiver unit 602 including several individual RF receiver modules communicates with several distinct transmitter units.

### DETAILED DESCRIPTION OF THE INVENTION

The catheter RF adapter of the present invention allows a diagnostic catheter to communicate wirelessly with an EP mapping system. Without a cable attaching the diagnostic catheter to an EP mapping system, a physician will be able to manipulate and control the catheter with greater ease.

The catheter RF adapter of the present invention comprises an RF transmitter module and a RF receiver module. The RF transmitter module is adapted to be securely attached to the handle of the catheter. The RF receiver module is coupled to the front end of the EP mapping system.

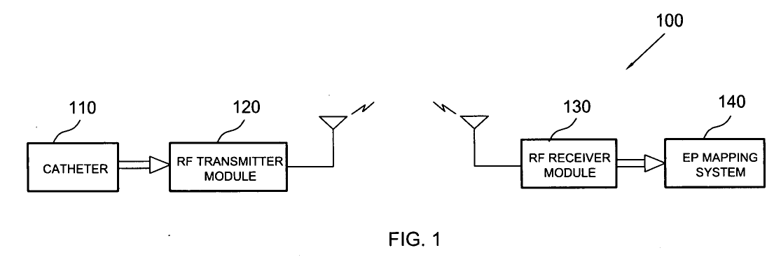

FIG. 1 is a block diagram illustrating the system 100 of the present invention. System 100 comprises a catheter 110, an RF transmitter module 120, an RF receiver module 130, and an EP mapping system 140.

The catheter 110 comprises a distal region. The catheter distal region includes bands of electrodes positioned spaced apart in different longitudinal sections of the distal region. The tip of the catheter may also include an electrode. The catheter tip electrode and the catheter bands of electrodes send electrocardiograph (ECG) signals to the RF transmitter module 120. The tip electrode and the number of bands of electrodes determine the number of signals being outputted to the RF transmitter module 120, which in turn determine the number of RF channels used for wireless transmission. In one embodiment of the invention, the catheter 110 outputs 20 signals to the RF transmitter module 120 which processes the 20 signals and transmits the processed signals in 20 corresponding RF channels. The catheter 110 also includes a reference band electrode located at a large distance from the last band of electrode that senses an ECG signal, i.e., the furthest band electrode from the catheter distal end.

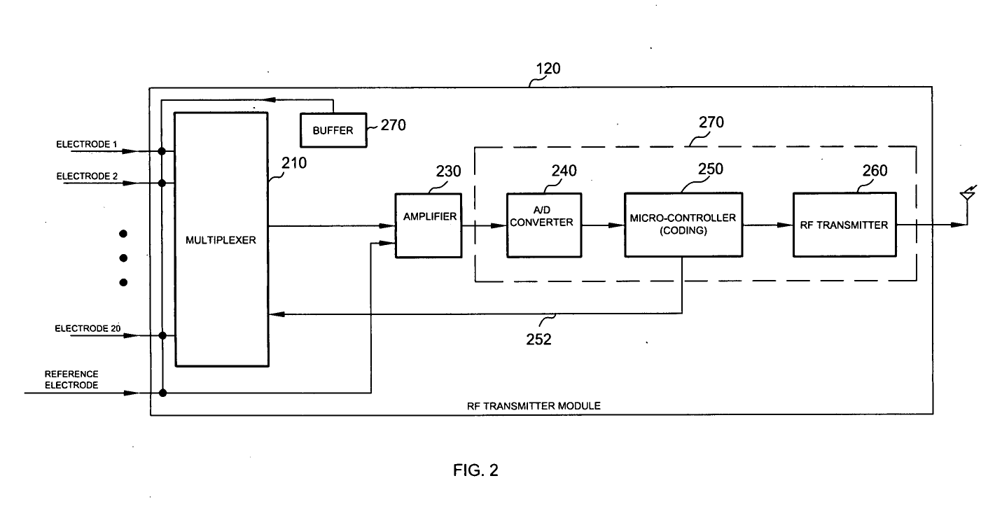

FIG. 2 is a block diagram of one embodiment of the RF transmitter module 120 of the present invention. In this embodiment, the RF transmitter module 120 comprises a multiplexer 210, an amplifier 230, an analog-to-digital (A/D) converter 240, a microcontroller 250, and an RF transmitter 260. The RF transmitter module 120 is securely attached to the handle of the catheter 110. The RF transmitter module 120 further comprises a buffer 270 to drive a DC voltage to each of the input signals to the multiplexer 210 and to the reference electrode of the catheter 110. Due to the buffer 270, the input signals to the multiplexer 210 and the signal from the reference electrode have each practically the same DC voltage component.

The multiplexer 210 receives, at its 20 inputs, 20 ECG analog signals in parallel from the catheter 110, and outputs a single ECG analog signal.

The amplifier 230 receives at its inputs the single ECG analog signal and the signal from the reference electrode. The amplifier 230 amplifies the difference between the ECG analog signal and the signal from the reference electrode to a level suitable for wireless transmission and outputs the amplified analog signal to the A/D converter 240.

The A/D converter 240 converts the amplified analog signal to a digital signal and outputs the digital signal to the microcontroller 250.

The microcontroller 250 codes the digital signal into a format suitable for wireless transmission. In one embodiment, an error correcting code is also employed in coding the digital signal. The microcontroller 250 output the coded digital signal to the RF transmitter 260. The microcontroller 250 also outputs a multiplexer control interface signal 252 to control the operation of the multiplexer 210.

The RF transmitter 260 receives the coded digital signal and transmits it over the air medium as an RF signal in a corresponding RF channel.

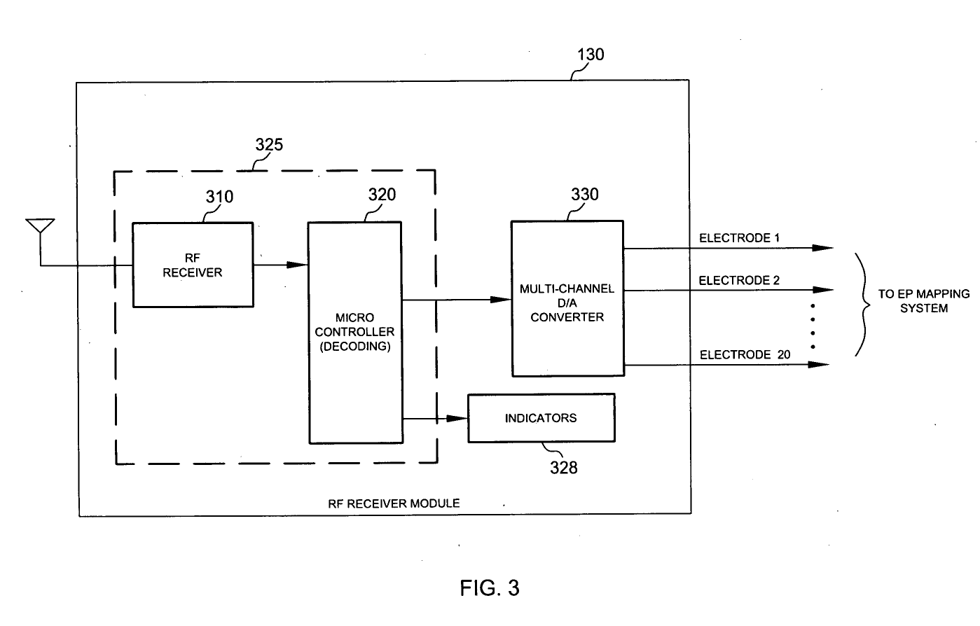

FIG. 3 is a block diagram of one embodiment of the RF receiver module 130 of the present invention. In this embodiment, the RF receiver module 130 comprises an RF receiver 310, a microcontroller 320, a multi-channel digital-to-analog (D/A) converter 330. The RF receiver module 130 also includes a set of indicators 328 to indicate status and any errors.

The RF receiver 310 receives the RF signal over the air medium from the corresponding RF channel and outputs the digital signal to the microcontroller 320.

The microcontroller 320 decodes the digital signal and outputs the decoded digital signal to the multi-channel D/A converter 330.

The multi-channel D/A converter 330 converts the digital signal into an analog signal. The multi-channel D/A converter 330 also demultiplexes the analog signal into 20 analog signals which are then outputted to the EP mapping system.

In order to measure the ECG signals, a reference signal is needed. In an existing catheter system where a catheter is connected to an EP system by a cable, a signal measured from a body surface of a patient via a patch connected directly to the EP system by a cable is used as a reference signal. In the present invention, where the communication to the EP system is wireless, a novel self-creating reference scheme is employed to provide a reference signal.

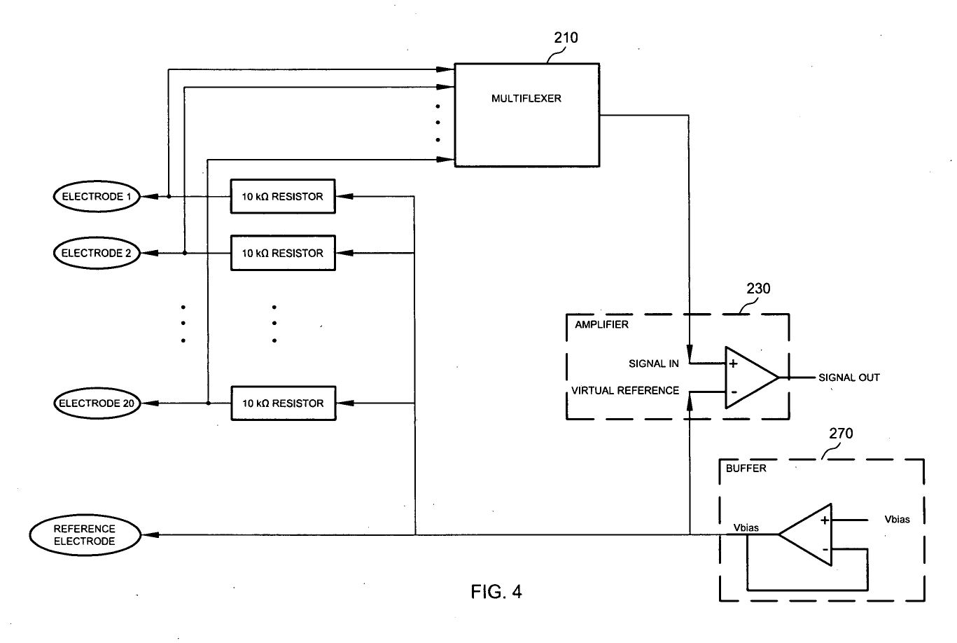

FIG. 4 is a block diagram illustrating the self-creating reference scheme of the present invention. Buffer 270, which comprises an operational amplifier configured as a voltage follower, drive a DC voltage V_{bias} to each of the electrodes of the catheter, including a reference electrode which is located on the catheter at a distance far from the last of the other electrodes. The distance is sufficiently large so that, when the distal portion of the catheter is placed inside the heart, the reference electrode is located outside and away from the heart. In one embodiment, the distance is 24 centimeters. In one embodiment, V_{bias} is about 1.5 Volts. Since the tissue impedance is about 100 Ohms to 120 Ohms, a 10 kilo-Ohms resistor is used for isolation for each of the ECG signals from the 20 electrodes.

Due to the buffer 270 driving the DC voltage V_{bias} out to the reference electrode, the signal from the reference electrode is practically equal to the DC voltage V_{bias}, and serves as a virtual reference.

The multiplexer 210 receives, at its 20 inputs, 20 ECG analog signals in parallel from the catheter 110, and outputs a single ECG analog signal.

The amplifier 230 comprises a differential amplifier. The differential amplifier receives the single ECG analog signal at its positive input and the signal from the reference electrode at its negative input. The amplifier 230 amplifies the difference between the 2 signals and outputs an amplified ECG analog signal that substantially does not have a DC component.

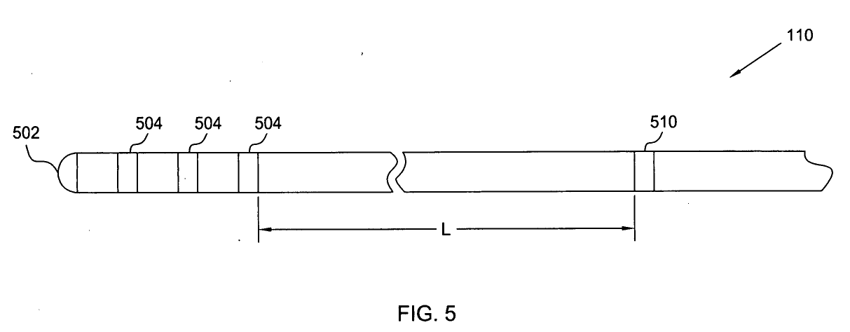

FIG. 5 shows an external view of an embodiment of the catheter 110. In this embodiment, the catheter 110 has a tip electrode 502 and 3 band electrodes 504 for detecting ECG signals. The catheter 110 further includes the reference band 510 for providing a reference signal to the amplifier 230. The reference band is located at a distance L from the last band electrode 504, that is, the furthest band electrode from the distal end of the catheter 110. In one embodiment, L is equal to 24 cm.

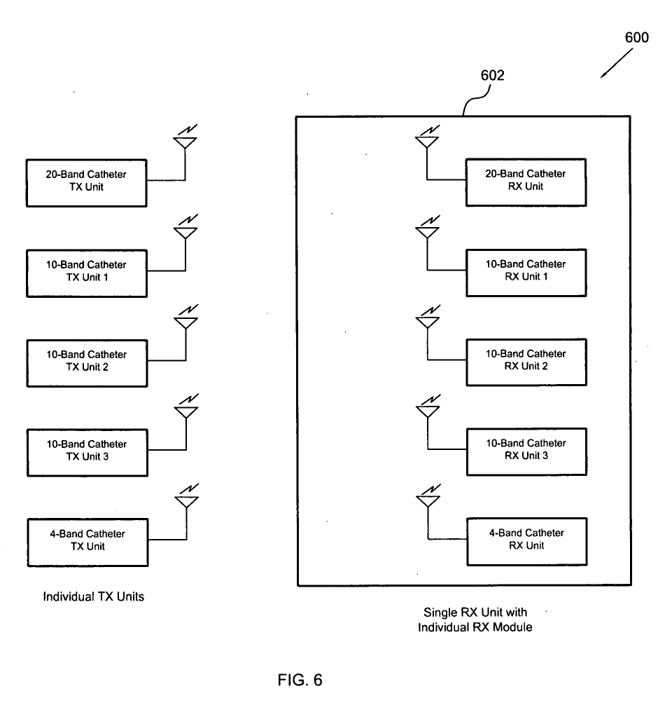

FIG. 6 shows an embodiment 600 of the system of the present invention, where a single receiver unit 602 including several individual RF receiver modules communicates with several distinct transmitter units. The single receiver unit 602 can identify the source of a received signal based on the specific RF channel frequency, data address packet, an unique identification of a transmitter unit. Software configuration is used to pair the transmitter catheter and the receiver unit. The transceivers can operates at frequencies between 2.400 GHz to 2.525 GHz.

While the invention has been described in terms of several embodiments, those of ordinary skill in the art will recognize that the invention is not limited to the embodiments described, but can be practiced with modification and alteration within the spirit and scope of the appended claims. The description is thus to be regarded as illustrative instead of limiting.

What is claimed is:

1. A catheter system for wireless communication with an electrophysiological (EP) mapping system, the catheter system comprising:
a catheter comprising:
an elongated body having a distal end, and a proximal end;
a plurality of mapping electrodes including a tip electrode being disposed on a distal portion of the elongated body, the plurality of mapping electrodes detecting electrocardiograph (ECG) signals; and
a reference electrode being disposed on the elongated body at a distance from the plurality of mapping electrodes such that the reference electrode substantially does not detect electrocardiograph (ECG) signals; and
a handle;
a catheter adapter attached to the handle, the catheter adapter including a radio frequency (RF) transmitter module for receiving, processing, and transmitting the detected ECG signals;
wherein the reference electrode provides a reference signal to the radio frequency (RF) transmitter module; and
an RF receiver module for receiving the transmitted ECG signals, the RF receiver module being coupled to the EP mapping system.
